(19) United States
(12) Patent Application Publication
(10) Pub. No.: US 2022/0176337 A1
(43) Pub. Date: Jun. 9, 2022

(54) SYSTEM AND METHOD USING PHOTOCHEMICAL OXYGEN STORAGE AND RELEASE

(71) Applicant: Portland State University, Portland, OR (US)

(72) Inventors: Theresa McCormick, Portland, OR (US); Luke Lutkus, Portland, OR (US)

(73) Assignee: Portland State University, Portland, OR (US)

(21) Appl. No.: 17/681,523

(22) Filed: Feb. 25, 2022

Related U.S. Application Data
(63) Continuation of application No. PCT/US2020/047829, filed on Aug. 25, 2020.
(60) Provisional application No. 62/892,758, filed on Aug. 28, 2019.

(51) Int. Cl. B01J 19/12; C01B 13/02

(52) U.S. Cl. CPC B01J 19/127; C01B 13/0211; B01J 19/128

(57) ABSTRACT

Disclosed herein is a method for converting light energy into mechanical energy and/or oxygen storage, purification, isolation, concentration, and/or removed. The method may comprise exposing a mixture of a polycyclic aromatic compound and a photosensitizer to oxygen and light to form an endoperoxide, and decomposing the endoperoxide to reform the polycyclic aromatic compound and oxygen. The polycyclic aromatic compound may be a naphthalene compound or anthracene compound and/or may have a formula

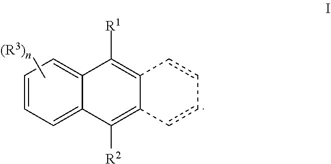

### CROSS REFERENCE TO RELATED APPLICATION

This application is a continuation of International Application No. PCT/US2020/047829, filed on Aug. 25, 2020, which was published in English under PCT Article 21(2), which in turn claims the benefit under 35 U.S.C. § 119(e) of the earlier filing date of U.S. Provisional Application No. 62/892,758, filed on Aug. 28, 2019, which is incorporated herein by reference in its entirety.

### FIELD

The present application concerns a system and method for oxygen storage and release and conversion of light into mechanical energy using a photochemical reaction.

### BACKGROUND

Oxygen is typically purified using energy intensive processes such as distillation of liquefied air, and storing the oxygen efficiently usually requires compressing to high pressures and/or liquefying. Oxygen also can be produced from certain irreversible reactions, such as from alkali perchlorates that are exposed to high temperatures, but this also may release toxic chlorine gas. Few chemically reversible systems for oxygen storage have been reported and they typically require high temperatures for oxygen release.

Additionally, few methods have been reported for converting light energy, such as solar energy, into mechanical energy. Mechanical energy has been generated from ultraviolet (UV) irradiation using nitrosyl chloride (NOCl) gas splitting and recombination reactions to produce NO and Cl₂. However, the maximum potential for this process is limited to a pressure/volume change of twice as much as the initial number of moles of NOCl and also requires UV light. Additionally, nitrosyl chloride is highly toxic, irritating to the eyes, lungs and skin, and is a strong oxidizing agent, and both of the reaction products, chlorine and NO gas, also are toxic.

### SUMMARY

Disclosed herein are embodiments of a system and method for converting light energy into mechanical energy and/or oxygen storage, purification, isolation, and/or concentration. Embodiments of the method may comprise exposing a first mixture of comprising a polycyclic aromatic compound and a photosensitizer to oxygen gas and light to form a second mixture comprising an endoperoxide; and changing a temperature of the second mixture, and/or stopping irradiation of the second mixture by the light, to regenerate at least a portion of the polycyclic aromatic compound and release at least a portion of the oxygen gas. In some embodiments, the method comprises exposing a first mixture comprising a polycyclic aromatic compound and a photosensitizer to a first portion of oxygen gas and light at a first temperature to form a second mixture comprising an endoperoxide, the first temperature being selected to substantially prevent the endoperoxide from decomposing. The method further comprises raising the temperature of the second mixture to a second temperature suitable to facilitate decomposition of the endoperoxide, form a regenerated polycyclic aromatic compound and release oxygen. Typically, the second temperature is higher than the first temperature. The method also comprises mixing the released oxygen with an air stream to form an oxygen enriched air stream that optionally can be provided to a patient in need thereof. Exposing the first mixture to oxygen gas may comprise exposing the first mixture to ambient air and/or the method may further comprise maintaining the second mixture at or below the first temperature for a selected time period before raising the temperature of the second mixture to the second temperature. Optionally, the method may comprise exposing the regenerated polycyclic aromatic compound and the photosensitizer to a second portion to oxygen gas to reform at least a portion of the endoperoxide.

An alternative embodiment of the method comprises a) exposing a first mixture comprising a first polycyclic aromatic compound and a first photosensitizer to a first portion of oxygen gas and light from a first light source at a first temperature to form a second mixture comprising a first endoperoxide, where the first temperature is selected to substantially prevent decomposition of the first endoperoxide;

b) raising the temperature of the second mixture to a second temperature higher than the first temperature and suitable to decompose the first endoperoxide thereby regenerating at least a portion of the first polycyclic aromatic compound and forming a second portion of oxygen gas that is mixed with a first air stream to form a first oxygen enriched air stream;

c) exposing a third mixture comprising a second polycyclic aromatic compound and a second photosensitizer to a third portion of oxygen gas and light from a second light source at a third temperature to form a fourth mixture comprising a second endoperoxide, where the third temperature is selected to substantially prevent decomposition of the second endoperoxide:

d) when the first endoperoxide in the second mixture has substantially decomposed, raising the temperature of the third mixture to a fourth temperature that is higher than the third temperature and selected to decompose the second endoperoxide, thereby regenerating at least a portion of the second polycyclic aromatic compound and forming a fourth portion of oxygen gas that is mixed with a second air stream to form a second oxygen enriched air stream; and

e) exposing the first polycyclic aromatic compound and the first photosensitizer to a fifth portion of oxygen gas and light from the first light source to reform at least a portion of the first endoperoxide. Optionally, steps b and c may be performed contemporaneously and/or steps d and e may be performed contemporaneously.

Additional embodiments of the method comprise exposing a first mixture comprising a polycyclic aromatic compound, a photosensitizer and oxygen gas to light in an enclosed space having a first volume to form an endoperoxide by a reaction between the polycyclic aromatic compound and the oxygen gas, thereby changing the volume of the enclosed space to a second volume that is less than the first volume. The method may further comprise removing the light to regenerate the polycyclic aromatic compound and release at least a portion of the oxygen gas into the enclosed space, thereby changing the volume of the enclosed space to a third volume that is greater than the second volume and may be substantially the same as the first volume. Changing the volume of the enclosed space may create mechanical work, such as causing a piston to move.

Further embodiments of the method comprise exposing a first mixture comprising a polycyclic aromatic compound, a photosensitizer and oxygen gas to light in an enclosed space having a first pressure to form an endoperoxide by a reaction between the polycyclic aromatic compound and the oxygen gas, thereby changing the pressure in the enclosed space to a second pressure that is less than the first pressure. The method may further comprise removing the light to regenerate the polycyclic aromatic compound and release at least a portion of the oxygen gas into the enclosed space, thereby changing the pressure of the enclosed space to a third pressure that is greater than the second pressure and may be substantially the same as the first volume. Changing the pressure in the enclosed space may result in mechanical work.

Embodiments of the disclosed method may further comprise exposing the regenerated polycyclic aromatic compound to light to reform at least a portion of the endoperoxide, and then removing the light source to regenerate the polycyclic aromatic compound a second time. Exposing the first mixture to light may be performed at a first temperature, and/or removing the light may comprise raising the temperature to a second temperature higher than the first temperature.

In any embodiments, removing the light and/or stopping or preventing irradiation by the light may comprise physically removing the light source from the mixture, physically removing the mixture from the light source, or a combination thereof; blocking the light source; tuming off the light source; or any combination thereof.

In any of the disclosed embodiments, the polycyclic aromatic compound may be a naphthalene compound or an anthracene compound, and/or may have a formula I

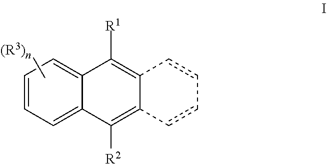

With respect to formula I, each of R¹, R² and R³ independently is H, OH, aliphatic, aryl, alkoxy, —O-acyl, —O—Si(alkyl)₃, —O-amino acid, or —O-carbohydrate; n is from 0 to 6; and “---” represents a bond that may or may not be present. In certain embodiments, compound has a formula selected from

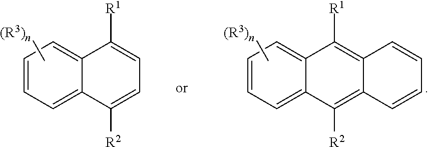

In some embodiments, n=) and/or at least one of R¹ and R² are not H. R¹ and R² both may be alkyl, and/or may be the same or different from each other. In certain embodiments, R¹ and R² are the same and not H. And in particular embodiments, the polycyclic aromatic compound is 1,4-dimethylnaphthalene.

Also, in any disclosed embodiments, the first temperature may be from −78° C. to 100° C., such as from 0° C. to 20° C. Additionally. or alternatively, the second temperature may be from −40° C. to 100° C. or more, such as from −40° C. to 50° C., or from 15° C. to 25° C.

The photosensitizer may be rose bengal, methylene blue, Eosin B, Ru(bpy)₃, methyl green, rubrene, a fullerene, a nanoparticle or a combination thereof, and in certain disclosed embodiments, the photosensitizer is rose bengal. Additionally, or alternatively, the light may be, or comprise, visible light and/or near infrared, and/or may have a wavelength of from 380 nm to 1000 nm, such as from 380 nm to 740 nm or from 530 nm to 575 nm. In any embodiments, the light may be, or comprise natural light, or the light may comprise light from an artificial light source, such as LED light.

In any embodiments, the first mixture may be a solution or a solid. In embodiments where the first mixture is a solution, the solution may further comprise a solvent. The solvent can be any suitable solvent such as acetonitrile, ethanol, methanol, dichloromethane, hexanes, water, ether, dimethylformamide, carbon tetrachloride, chloroform, propylene carbonate, ethylene glycol, propylene glycol, tetrahydrofuran or a combination thereof. In alternative embodiments where the first mixture is a solid, the solid may further comprise a solid support, such as a zeolite, silica such as diatomaceous earth, hydrogel, polymer, metal-organic framework motif, or a combination thereof. In certain embodiments, the polycyclic aromatic compound and/or the photosensitizer compound are associated with, such as absorbed, adsorbed or covalently attached to a polymer that forms at least part of the solid support. The polymer may be a polyalkyl polymer, such as polyethylene or polypropylene; a polyalkyl glycol, such as polyethylene glycol, or polypropylene glycol; a polypeptide; a conjugated polymer chain; a polyacrylic acid, or a combination thereof. In some embodiments, the polycyclic aromatic compound and/or the photosensitizer are covalently attached to the polymer, and in certain embodiments, both the polycyclic aromatic compound and the photosensitizer are covalently attached to the polymer. In some embodiments, the polymer has a molecular weight of from 3 kDa or less to 1000 kDa or more, such as from 5 kDa to 50 kDa. As used herein, the term “molecular weight” with respect to a polymer is a weight averaged molecular weight determined by proton NMR and size exclusion chromatography.

Embodiments of a system suitable to perform the disclosed method also are disclosed herein. The system may comprise a first chamber comprising a first mixture comprising a first polycyclic aromatic compound and a first photosensitizer, a first gas inlet, a first gas outlet, a first pump, and a first light source; a second chamber comprising a second mixture of a first polycyclic aromatic compound and a second photosensitizer, a second gas inlet, a second gas outlet, a second pump, and a second light source; and a controller configured to switch an air stream between the first and second gas inlets. The system also may comprise a temperature controller, such as a heater, a cooler, or a combination thereof.

Alternative embodiments of the system concern an enclosed space that can vary in volume and/or pressure, and comprises a mixture comprising a polycyclic aromatic compound, a photosensitizer, and a gas comprising oxygen, and optionally a solvent. The system may further comprise a light source.

The foregoing and other objects, features, and advantages of the disclosure will become more apparent from the following detailed description, which proceeds with reference to the accompanying figures.

### BRIEF DESCRIPTION OF THE DRAWINGS

FIG. 1 is a graph of absorbance versus wavelength, illustrating the absorbance profiles of various commercially available triplet photosensitizers.

FIG. 2 is a graph of change of pressure (PSI) versus time, illustrating the changes in pressure over time resulting from the consumption of O₂ under green light irradiation and subsequent release of oxygen at 22° C. for a mixture of 1,4-dimethylnaphthalene and rose bengal.

FIG. 3 is a graph of change of pressure (PSI) versus time, illustrating the changes in pressure over time resulting from cycling the lights on and off every four hours at 25° C. under atmospheric conditions.

FIG. 4 is a graph of change in pressure (PSI) versus time, illustrating the changes in pressure over time resulting from cycling the lights on and off every six hours at 25° C. under atmospheric conditions.

FIG. 5 is a graph of change of pressure (PSI) versus time, illustrating the pressure change over time of 20%, 30%, and 50% 1,4-dimethylnaphthalene (1,4-DMN) in an oxygen atmosphere irradiated with green light.

FIG. 6 is a schematic diagram of an exemplary embodiment of a system for converting light energy into mechanical energy through pressure changes due to oxygen uptake and release.

FIG. 7 is a schematic diagram of an exemplary embodiment of a continuous flow system for oxygen purification, concentration and/or isolation.

FIG. 8 is a schematic diagram of an exemplary embodiment of an oxygen concentrator.

FIG. 9 is a graph of change of pressure (PSI) versus time, illustrating the pressure changes resulting from irradiation of 1,4-DMN and rose bengal for 90 minutes followed by 180 minutes without irradiation, at 25° C. under an oxygen headspace for three cycles.

### DETAILED DESCRIPTION

### I. Definitions

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

“Substituted,” when used to modify a specified group or moiety, means that at least one, and perhaps two or more, such as 2, 3, 4, or more, hydrogen atoms of the specified group or moiety is independently replaced with the same or different substituent groups as defined below, and as allowed by valency rules. In any particular embodiment, a group, moiety or substituent may be substituted or unsubstituted, unless expressly defined as either “unsubstituted” or “substituted.” Accordingly, any of the groups specified herein may be unsubstituted or substituted. In particular embodiments, the substituent may or may not be expressly defined as substituted, but is still contemplated to be optionally substituted. For example, an “alkyl” or an “aryl” moiety may be unsubstituted or substituted, but an “unsubstituted alkyl” or an “unsubstituted aryl” is not substituted. Exemplary substituents include, but are not limited to, OH, halogen, such as F, Cl, B, or I; alkyl, such as methyl, ethyl, propyl, isopropyl, butyl, iso-butyl, sec-butyl, or tert-butyl; alkoxy; acyl; benzyl; aryl, such as phenyl or napthyl; or a combination thereof.

“Acyl” refers to the group —C(O)R, where R is H, aliphatic, or aromatic. Exemplary acyl moieties include, but are not limited to, —C(O)H, —C(O)alkyl, —C(O)C₁-C₆alkyl, —C(O)cycloalkyl, —C(O)alkenyl, —C(O)cycloalkenyl, —C(O)alkynyl, or —C(O)aryl. Specific examples include —C(O)H, —C(O)Me, —C(O)Et, or —C(O)phenyl.

“Aliphatic” refers to a substantially hydrocarbon-based group or moiety. An aliphatic group or moiety can be acyclic, including alkyl, alkenyl, or alkynyl groups, cyclic versions thereof, such as cycloaliphatic groups or moieties including cycloalkyl, cycloalkenyl or cycloalkynyl, and further including straight- and branched-chain arrangements, and all stereo and position isomers as well. Unless expressly stated otherwise, an aliphatic group contains from one to twenty-five carbon atoms (C₁₋₂₅); for example, from one to fifteen (C₁₋₁₅), from one to ten (C₁₋₁₀), from one to six (C₁₋₆), or from one to four carbon atoms (C₁₋₄) for a saturated acyclic aliphatic group or moiety, from two to twenty-five carbon atoms (C₂₋₂₅); for example, from two to fifteen (C₂₋₁₅), from two to ten (C₂₋₁₀), from two to six (C₂₋₆), or from two to four carbon atoms (C₂₋₄) for an unsaturated acyclic aliphatic group or moiety, or from three to fifteen (C₃₋₁₅) from three to ten (C₃₋₁₀), from three to six (C₃₋₆), or from three to four (C₃₋₄) carbon atoms for a cycloaliphatic group or moiety. An aliphatic group may be substituted or unsubstituted, unless expressly referred to as an “unsubstituted aliphatic” or a “substituted aliphatic,” or, for example, unsubstituted alkyl, alkenyl or alkynyl, or cyclic versions thereof, or substituted alkyl, alkenyl or alkynyl, or cyclic versions thereof. An aliphatic group can be substituted with one or more substituents (up to two substituents for each methylene carbon in an aliphatic chain, or up to one substituent for each carbon of a —C═C— double bond in an aliphatic chain, or up to one substituent for a carbon of a terminal methine group).

“Alkoxy” refers to the group —OR, where R is a substituted or unsubstituted alkyl or a substituted or unsubstituted cycloalkyl group. In certain examples R is a C₁₋₆ alkyl group or a C₃₋₆cycloalkyl group. Methoxy (—OCH₃) and ethoxy (—OCH₂CH₃) are exemplary alkoxy groups. In a substituted alkoxy, R is substituted alkyl or substituted cycloalkyl, examples of which include haloalkoxy groups, such as —OCF₂H, or —OCF₃.

“Alkyl” refers to a saturated aliphatic hydrocarbyl group having from 1 to 25 (C₁₋₂₅) or more carbon atoms, more typically 1 to 10 (C₁₋₁₀) carbon atoms such as 1 to 6 (C₁₋₆) carbon atoms or 1 to 4 (C₁₋₄) carbon atoms. An alkyl moiety may be substituted or unsubstituted. This term includes, by way of example, linear and branched hydrocarbyl groups such as methyl (CH₃), ethyl (—CH₂CH₃), n-propyl (—CH₂CH₂CH₃), isopropyl (—CH(CH₃)₂), n-butyl (—CH₂CH₂CH₂CH₃), isobutyl (—CH₂CH₂(CH₃)₂), sec-butyl (—CH(CH₃)(CH₂CH₃), t-butyl (—C(CH₃)₃), n-pentyl (—CH₂CH₂CH₂CH₂CH₃), and neopentyl (—CH₂C(CH₃)₃).

“Aromatic” refers to a cyclic, conjugated group or moiety of, unless specified otherwise, from 5 to 15 ring atoms having a single ring (e.g., phenyl, pyridinyl, or pyrazolyl) or multiple condensed rings in which at least one ring is aromatic (e.g., naphthyl, indolyl, or pyrazolopyridinyl), that is at least one ring, and optionally multiple condensed rings, have a continuous, delocalized n-electron system. Typically, the number of out of plane π-electrons corresponds to the Hückel rule (4n+2). The point of attachment to the parent structure typically is through an aromatic portion of the condensed ring system. For example,

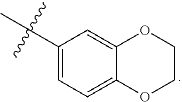 [SMILES: *c1ccc2c(c1)OCCO2]

However, in certain examples, context or express disclosure may indicate that the point of attachment is through a non-aromatic portion of the condensed ring system. For example,

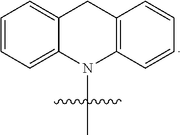 [SMILES: *N1c2ccccc2Cc2ccccc21]

An aromatic group or moiety may comprise only carbon atoms in the ring, such as in an aryl group or moiety, or it may comprise one or more ring carbon atoms and one or more ring heteroatoms comprising a lone pair of electrons (e.g. S, O, N, P, or Si), such as in a heteroaryl group or moiety. Unless otherwise stated, an aromatic group may be substituted or unsubstituted.

“Aryl” refers to an aromatic carbocyclic group of, unless specified otherwise, from 6 to 15 carbon atoms having a single ring (e.g., phenyl) or multiple condensed rings in which at least one ring is aromatic multiple condensed rings in which at least one ring is aromatic (e.g., 1,2,3,4-tetrahydroquinoline, benzodioxole, and the like) providing that the point of attachment is through an aromatic portion of the ring system. If any aromatic ring portion contains a heteroatom, the group is heteroaryl and not aryl. Aryl groups may be, for example, monocyclic, bicyclic, tricyclic or tetracyclic. Unless otherwise stated, an aryl group may be substituted or unsubstituted.

### II. Overview

Certain polycyclic aromatic compounds, such as an optionally substituted naphthalene compound or anthracene compound, can react with singlet oxygen to reversibly form an endoperoxide. As used herein, the terms naphthalene compound and anthracene compound include the parent compounds, namely naphthalene or anthracene respectively, unless expressly excluded or if the context dictates otherwise. Scheme 1 illustrates an exemplary reversible reaction of 1,4-dimethylnapthalene (1,4-DMN) to produce the corresponding 1,4-DMN endoperoxide. Singlet oxygen (¹O₂) is an excited state of oxygen with unique reactivity compared to that of ground state oxygen, known as triplet oxygen (³O₂). The unique reactivity of O₂ has found utility for organic oxidations and cancer treatments. The stability of substituted naphthalene and anthracene endoperoxides may vary with different groups at the 1 and 4 positions of the naphthalene, or 9 and positions of the anthracene. Endoperoxides that are less stable at a particular temperature will break down more easily to release ¹O₂ and regenerate the original naphthalene or anthracene compound than more stable endoperoxides.

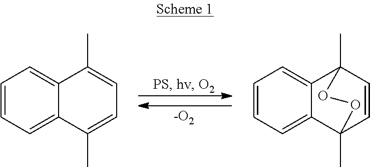

The formation of an endoperoxide, such as a 1,4-DMN endoperoxide, results from a reaction with singlet oxygen that can be produced using triplet photosensitizers (PS), which transfer energy to ground state oxygen upon suitable visible light irradiation (Scheme 1). Many triplet photosensitizers are commercially available, and the suitability of any particular photosensitizer is only limited by solubility, as long as it does not react with ¹O₂, the polycyclic aromatic compound, or the resulting endoperoxide. As such, nearly the entire visible spectrum and NIR may be used to create ¹O₂ and therefore the endoperoxide by selecting a particular photosensitizer or combination of photosensitizers (FIG. 1). Suitable photosensitizers include, but are not limited to, rose bengal, methylene blue, Eosin B, Ru(bpy)₃, methyl green, rubrene, a fullerene (alkyl, aryl, alkoxy-substituted or unsubstituted C₂₀₋₉₄) a fluorene (e.g. comprising 9,9-substituted, or 2,7-substituted fluorene) a nanoparticle (e.g. CdTe. ZnSe, SiNP, CNP. AuNP, BiNP, or nanoparticles comprising encapsulated small molecule triplet photosensitizers that are attached or encapsulated by silica, proteins, or polymers), or a combination thereof.

Typical solar energy conversion reactions transform solar energy into electricity that has to be stored until needed. In artificial photosynthesis, solar energy is transformed into chemical potential in the form of a solar fuel that can be stored until needed. In contrast, disclosed herein are embodiments of a system and method that harnesses the reaction of polycyclic aromatic compounds with singlet oxygen for use in energy or oxygen storage and release applications. Certain embodiments allow solar energy to be directly transformed into mechanical energy by producing a pressure and/or volume change in the system that can drive a mechanical device, such as a piston. Other embodiments allow for oxygen to be reversibly removed from a gas mixture, leading to alternative applications, such as oxygen storage, purification, concentration and/or removal.

The endoperoxide that is formed by the reaction of the naphthalene or anthracene compound and singlet oxygen releases oxygen at a temperature dependent rate. Therefore, the consumption of oxygen can be controlled by controlling the exposure to light, while the oxygen release can be controlled using heat. Such a system and method shows promise for the generation of mechanical work in the form of pressure or volume change from light. To the inventors' knowledge direct conversion of visible light to mechanical energy has not been previously reported. Furthermore, due to the selective reaction with singlet oxygen this approach can be used for the purification and/or removal of oxygen from gaseous mixtures such as atmospheric air.

### III. Compounds

Compounds suitable for use in the disclosed technology include aromatic compounds that can form an endoperoxide with singlet oxygen. In some embodiments, the compound is a polycyclic aromatic compound comprising two or more rings, such as 2, 3, 4, 5 or more aromatic rings, and preferably two or three aromatic rings. Certain embodiments concern a compound having a formula I

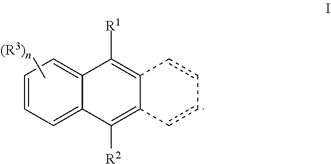

With respect to formula I, the dashed lines ( [symbol] ) indicate optional bonds, i.e., the compound is either a naphthalene compound having a formula II

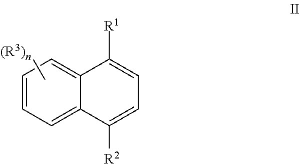

or an anthracene

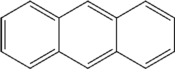 [SMILES: c1ccc2cc3ccccc3cc2c1]

compound having a formula III

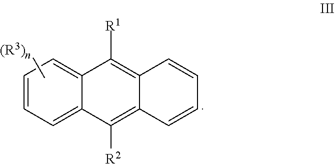

Also with respect to formulas I, II and III, each of R and R² independently is H; OH; aliphatic, such as alkyl (for example, methyl, ethyl, iso-propyl, or n-propyl), alkenyl (for example, ethenyl or propenyl) or alkynyl (for example, ethynyl or propynyl) or cyclic versions thereof; aromatic, such as aryl, (for example phenyl or naphthyl); —CH₂aryl, such as benzyl; alkoxy, such as —O-alkyl, preferably —O—CH₃, —O-Et, or —O-iPr; —O-acyl, such as —O—C(O)alkvl or —O—C(O)aryl, preferably, —O—C(O)CH₃ or —O—C(O)phenyl; —O—Si(alkyl)₃, such as —O—Si(Me)₃ or —O—Si(iPr)₃; halogen, such as F. Cl, Br, or I; cyano; —O-amino acid, such as —O-Glu; —O-carbohydrate, such as

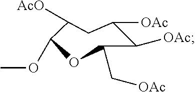

or a polymer. n is from 0 to 6, such as 0, 1, 2, 3, 4, 5, or 6. And R³, if present, is OH; aliphatic, such as alkyl (for example, methyl, ethyl, iso-propyl, or n-propyl), alkenyl (for example ethenyl or propenyl) or alkynyl (for example, ethynyl or propynyl) or cyclic versions thereof; aromatic, such as aryl, (for example, phenyl); alkoxy, such as —O-alkyl, preferably —O—CH, —O-Et, or —O-iPr; —O-acyl, such as —O—C(O)alkyl or —O—C(O)aryl, preferably, —O—C(O)CH₃ or —O—C(O)phenyl; —O—Si(alkyl)₃, such as —O—Si(Me)₃ or —O—Si(iPr)₃; halogen, such as F, Cl, Br, or I; cyano; —O-amino acid, such as —O-Glu; or —O-carbohydrate, such as

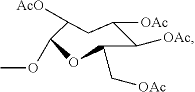

or a polymer.

Each polymer, if present, may be independently a polyalkyl polymer, such as polyethylene or polypropylene; a polyalkyl glycol, such as polyethylene glycol, or polypropylene glycol; a polypeptide; a polyacrylic acid polymer, or a conjugated polymer chain. In certain embodiments, the photosensitizer also may be conjugated to the polymer.

In some embodiments, R¹ and R² are the same, but in other embodiments, R¹ and R² are different. In some embodiments, at least one of R¹ and R² is not H. and in certain embodiments, both of R¹ and R² are not H.

In certain embodiments, n=0.

In some embodiments, n=0 and R¹ and R² are both alkyl, such as methyl. In particular embodiments, the compound is 1,4-dimethylnaphthalene (1,4-DMN).

### IV. Oxygen Uptake and Release

Oxygen uptake is facilitated by irradiation with light of a mixture of the polycyclic aromatic compound, such as naphthalene and/or anthracene, or a derivative thereof, and a photosensitizer. The mixture may be a solution comprising the polycyclic aromatic compound and photosensitizer in a suitable solvent. Solvents suitable for use in the disclosed technology include any solvent that will dissolve the polycyclic aromatic compound, such as a naphthalene and/or anthracene compound, and photosensitizer while not chemically reacting with the polycyclic aromatic compound, the photosensitizer, the singlet oxygen or the resulting endoperoxide. Suitable solvents include, but are not limited to, acetonitrile; chlorinated solvents, such as dichloromethane, chloroform, dichloroethane carbon tetrachloride, and chloroform; propylene carbonate; ethylene glycol; propylene glycol; tetrahydrofuran; hexanes; ether; dimethylformamide; water; ethanol; methanol; or deuterated analogs thereof; or a combination thereof.

In alternative embodiments, the disclosed system and method may comprise a solid-state polycyclic aromatic compound/photosensitizer composition. Adapting to a solid-state system may reduce or eliminate the need for bubbling or mixing a gas, such as air, to promote oxygen dissolution. In such a solid-state system, the photosensitizer and the polycyclic aromatic compound, such as 1,4-DMN, are loaded onto a suitable solid-state support, such as by adsorption, absorption, and/or attached through a covalent or ionic bond. Suitable solid-state supports include, but are not limited to, porous and/or gas permeable materials, such as zeolites, silica such as diatomaceous earth, hydrogels, polymers, metal-organic framework motif, or a combination thereof. The solid support may be selected to be transparent to a wavelength of light suitable to excite the triplet photosensitizer, for example, diatomaceous earth at visible-NIR wavelengths. And/or the pathlength of the solid support may be selected to increase the transparency, for example, with polymers, and/or to have a depth comparable with the maximum depth of penetration of the light. Additionally, or alternatively, the solid support may absorb the light and act as a singlet oxygen photosensitizer, for example, with nanoparticles.

The light may be polychromic or monochromic visible and/or near infrared light and/or may have or comprise a wavelength of from 380 nm or less to 1000 nm or more, such as from 380 nm to 740 nm, from 380 nm to 700 nm. In certain embodiments, the light has or comprises a wavelength of from 500 nm to 600 nm, such as 530 nm to 575 nm. The wavelength(s) of light may be selected based on the absorbance of the photosensitizer(s). Rose Bengal may be excited by green light from 500-570 nm or methylene blue may be excited using red light from 580-640 nm. The light may be natural light, such as sunlight, and may be used unfiltered or with filters for the only the desired wavelength ranges. Additionally, or alternatively, the light may comprise artificial light, such as LED light, that optionally is selected to provide light having a desired wavelength(s).

In some embodiments of the disclosed system and method, oxygen uptake and oxygen release typically are performed in separate steps. Each step is performed at a temperature suitable to facilitate the respective reaction. For example, oxygen uptake may be performed at a temperature of from −78° C. or lower to 100° C. or higher, such as from −78° C. to 50° C., from −78° C. to 20° C., from −50° C. to 20° C., from −25° C. to 20° C., from 0° C. to 20° C. or from 10° C. to 20° C. And oxygen release may proceed at a temperature from −40° C. to 100° C. or more, such as from −20° C. to 50° C., from −20° C. to 30° C., from −10° C. to 30° C., from 0° C. to 30° C., from 10° C. to 30° C., from 15° C. to 25° C. or from 20° C. to 25° C.

In some embodiments, oxygen release is performed at a temperature greater than the temperature at which oxygen uptake occurs. In other embodiments, the temperatures at which the two reactions are performed may overlap or be substantially the same, but when the mixture of the polycyclic aromatic compound and photosensitizer is being irradiated, oxygen uptake dominates oxygen release, leading to an overall increase in the concentration of endoperoxide and a decrease in the concentration of oxygen gas. In such embodiments, once irradiation is stopped oxygen release is dominant, and thus results in an increase in the oxygen gas concentration.

In some embodiments, the endoperoxide can be stored at a temperature below the oxygen release temperature thereby storing the oxygen that has been removed from the gas. The endoperoxide can be stored for a desired time period, such as from greater than zero to one year or more, such as from greater than zero to 52 weeks, from greater than zero to 30 weeks, from greater than zero to 20 weeks, from greater than zero to 10 weeks, from greater than zero to 4 weeks, from greater than zero to 1 week, from greater than zero to 5 days, from greater than zero to 3 days, from greater than zero to 2 days from greater than zero to 24 hours, from greater than zero to 18 hours, from greater than zero to 12 hours, from 1 hour to 6 hours, or from 1 hour to 3 hours. In some embodiments, the endoperoxide is used to store oxygen for emergency situations, such as for use in airplane emergency oxygen systems.

For a preliminary study, rose bengal was used as the photosensitizer because of its availability and high ¹O₂ quantum yield (φ≈0.76). In a closed system, the photocatalytic oxidation of 1,4-DMN (6.4×10⁻² M) with rose bengal (1×10⁻⁴ M) in acetonitrile (10 mL) under green light irradiation was monitored by the corresponding pressure decrease from oxygen consumption. The stability of the corresponding endoperoxide varied with temperature. Upon turning off the green light irradiation, ¹O₂ was no longer generated and the 1,4-DMN endoperoxide was able to be stored at temperatures of 20° C. or below.

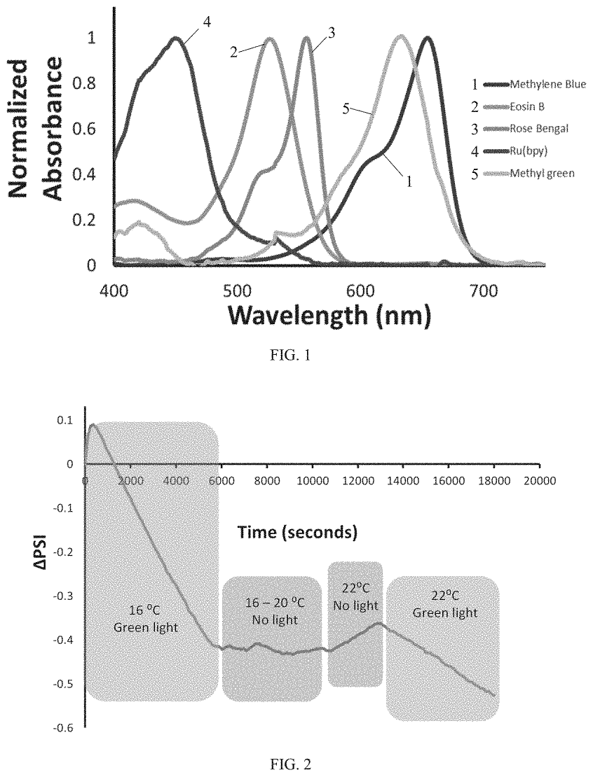

When the temperature was raised to above 20° C. oxygen was released and the 1,4-DMN was regenerated. The release of oxygen was monitored by a corresponding increase in pressure. As shown in FIG. 2, the initial generation of ¹O₂ resulted in the formation of the endoperoxide and a decrease in the pressure of the system due to oxygen consumption by 1,4-DMN. The results also showed that the endoperoxide was relatively stable between 16° C. and 20° C. as no pressure increase was observed at these temperatures when the green light irradiation was turned off.

As the temperature was raised to 22° C., a pressure increase was observed, likely due to the destabilization of the endoperoxide and resulting oxygen release and 1,4-DMN regeneration. Upon green light irradiation at 22° C. the rate of oxidation dominated the oxygen release, resulting in an overall consumption of oxygen and decrease in pressure.

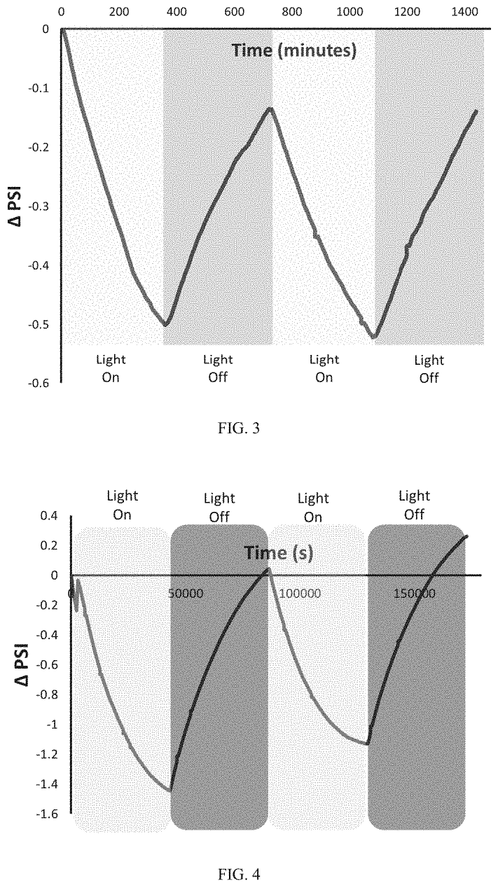

Because the photosensitizer does not chemically react or substantially degrade during the consumption and release of oxygen, and the polycyclic aromatic compound is regenerated, the system can be cycled repeatedly without a substantial loss of efficacy. This was demonstrated using 1,4-DMN over the course of two cycles at 25° C. by turning the green LEDs on and off in four-hour intervals (FIG. 3). The production of ¹O₂ from excitation of rose bengal resulted in a pressure decrease of about 0.5 PSI after four hours. After turning the lights off for four hours, the pressure returned to close to the starting pressure due to the release of stored oxygen. A similar pressure decrease/pressure increase pattern was observed during a second four-hour on/four-hour off irradiation cycle (FIG. 3).

Scaling up the amount of 1,4 DMN to 0.32 M yielded a similar result when cycled but with a pressure change of nearly 1.5 PSI over 12-hour intervals (FIG. 4). The same rate of pressure change was observed at both 1,4-DMN concentrations suggesting that oxygen is the limiting reagent. Increasing the amount of 1,4-DMN to 50% (3.2 M) resulted in a diminished rate of oxygen consumption and no oxygen consumption was observed at 90% (5.8 M) 1,4-DMN. The results indicated that at increasing concentrations of 1,4-DMN, the solubility of the oxygen in the 1,4-DMN solution may be decreasing, thereby reducing the effectiveness of the system. In some embodiments, the concentration of the polycyclic aromatic compound is from greater than zero to 4 M or more, such as from greater than zero to 3.5 M, from 0.01 M to 3 M, from 0.02 M to 2 M, from 0.05 M to 1 M or from 0.05 M to 0.75 M. In certain embodiments, such as at 25° C. under atmospheric conditions, a concentration range of 1,4-DMN was from 0.064 or less to 1 M or more, such as from 0.064 M to 0.64 M

One option to improve the oxygen consumption at higher 1,4-DMN concentrations is to increase the concentration of oxygen, such as by using substantially pure oxygen gas. Nearly equal rates of oxygen consumption were observed for 20%, 30%, and 50% (1.3 M, 2.0 M, and 3.2 M) solutions under an oxygen headspace (16.9 PSI, 1.13 atm) (FIG. 5). While high rates of oxygen consumption were observed, the nearly identical rates still indicated that 1,4-DMN did not limit the rate of oxygen consumption at these concentrations. However, for oxygen purification applications under atmospheric conditions, lower concentrations, such as 0.064 M or less to 1M, or from 0.064 M to 0.64 M of 1,4-DMN, were better suited in order to dissolve oxygen in the solution at 25° C. in certain solvents, such as acetonitrile.

The present system shows a number of significant advantages compared to many light energy systems, including solar energy systems. Since the role of the triplet photosensitizer is only to produce ¹O₂, a wide variety of dyes can be used to absorb across the visible spectrum including NIR light. Also, the efficiency increases at lower energy irradiation because each photon absorbed is available for the same goal of producing the endoperoxide. Furthermore, the reverse reaction does not require light to release oxygen and therefore such a system could be used for oxygen release at nighttime which is a major challenge for most solar energy technologies. While photothermal techniques have been used to transform solar energy into thermal energy, the disclosed system also can be cycled at a constant temperature by providing and removing the light. The disclosed exemplary system produced a quantum yield of endoperoxide formation around 0.34, which results in an efficiency of conversion to PV work of about 2% using 550 nm light.

### V. Applications

Embodiments of the disclosed system and method are useful for many applications, such as converting solar and/or artificial light into mechanical work, and/or oxygen purification, concentration, isolation, storage and/or removal.

A. Producing Mechanical Work

In an enclosed system, the pressure changes resulting from cycled on-off irradiation can result in change of volume to drive a mechanical system. In isothermal conditions the amount of work (W) from the system can be calculated from Equation 1

$$W = - nRT ln \frac{V 2}{V 1} . \qquad Equation 1$$

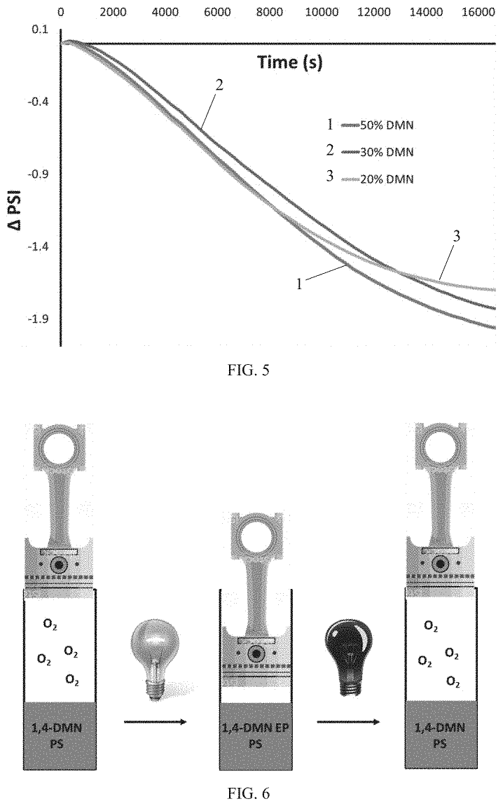

With respect to Equation 1, n is the number of moles of O₂, R is the ideal gas law constant, T is the temperature, V2 is the final volume and V1 is the initial volume. The change in volume could be used to drive a piston as demonstrated in FIG. 6. With respect to FIG. 6, a solution containing a triplet photosensitizer and a polycyclic aromatic compound, such as 1,4-DMN, removes oxygen from the gas in the headspace upon exposure to a suitable light source, resulting in a decrease in volume that can cause a piston to lower. When the light is removed or turned off, the solution releases oxygen back into the headspace to restore the initial volume and raise the piston. The overall result is mechanical work generation from cycling exposure to light. This can be done at one temperature where under irradiation O₂ uptake dominates O₂ release, alternatively the system could be cooled during irradiation to decrease the rate of O₂ release, and shielded from irradiation, or otherwise prevented from being irradiated, to increase the rate of O₂ release.

Mechanical energy has been generated using nitrosyl chloride (NOCl) gas splitting and recombination to produce NO and Cl₂. However, the maximum potential for this process is limited to a pressure/volume change of twice as much as the initial number of moles of NOCl and also requires UV light, as opposed to visible light, to operate. Additionally, nitrosyl chloride is highly toxic, irritating to the eyes, lungs and skin, and is a strong oxidizing agent. And both chlorine and NO gas also are toxic.

In contrast, the system and method disclosed herein has a number of advantages over previous attempts to produce mechanical work from light. First, the disclosed process can use ambient air, air that is enriched with oxygen, or a gas with a high oxygen concentration, such as substantially pure oxygen gas. As such, the potential environmental risks are significantly reduced. And since the role of the triplet photosensitizer is only to produce ¹O₂, a wide variety of dyes can be used to absorb across the visible spectrum including NIR light. Additionally, efficiency increases at lower energy irradiation because each photon absorbed is used to produce an endoperoxide. Furthermore, the release reaction does not require light to release oxygen and therefore such a system could be used at nighttime which is a major challenge for most solar energy technologies. While photothermal techniques have been used to transform solar energy into thermal energy, the disclosed process can be cycled at a constant temperature.

B. Continuous Flow Reactor

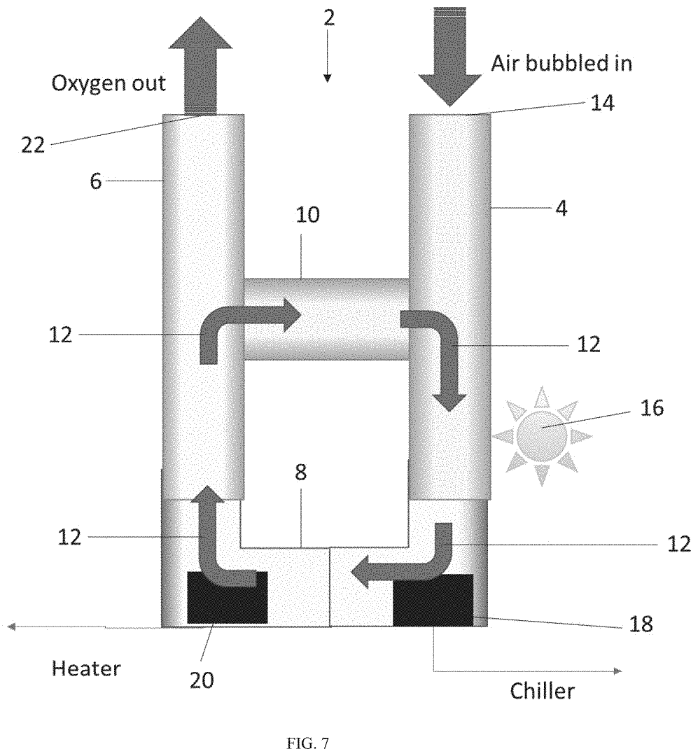

Additionally, disclosed herein are embodiments of a continuous flow reactor suitable for oxygen purification, concentration, storage, isolation and/or removal, such as from the atmosphere or from an alternative mixture of gases. FIG. 7 provides a schematic diagram illustrating an exemplary embodiment of a continuous flow reactor. With respect to FIG. 7, reactor 2 comprises a first reaction tube 4 and a second reaction tube 6. Reactor 2 also comprises a first connecting tube 8 and a second connecting tube 10 that fluidly couple first reaction tube 4 and second reaction tube 6 such that a fluid can flow in a loop as indicated by arrows 12, or in the opposite direction. The fluid may be a solution comprising a polycyclic aromatic compound, such as a naphthalene and/or anthracene compound, and a photosensitizer, optionally in a suitable solvent as disclosed herein.

Reactor 2 also comprises gas inlet 14 that facilitates a gas, such as air, being introduced into first reaction tube 4. In some embodiments, the gas is bubbled into the fluid in first reaction tube 4. In first reaction tube 4, the fluid, such as the solution comprising a polycyclic aromatic compound and a photosensitizer, is exposed to light 16 having a wavelength suitable to facilitate oxygen uptake from the gas to the polycyclic aromatic compound to form an endoperoxide. Light 16 may be natural light and/or artificial light, such as from an LED light source. FIG. 7 shows the light source being outside first reaction tube 4, but a person of ordinary skill in the art will appreciate that the light source could be located within first reaction tube 4 and/or be located in the walls of first reaction tube 4. For external light sources, such as shown in FIG. 7, first reaction tube 4 may comprise a material that facilitates the passage of light from the light source to the fluid. In some embodiments, first reaction tube 4 may be partially or substantially completely formed from the material, but in other embodiments, first reaction tube 4 comprises one or more windows that facilitate light passage. Suitable materials include, but are not limited to, glass, acrylic, polyvinylidiene chloride, polycarbonate, silica, and combinations thereof.

Reactor 2 also may comprise chiller 18 that lowers and/or maintains the temperature of the fluid at a first temperature suitable to facilitate oxygen uptake and/or endoperoxide formation. FIG. 7 shows optional chiller 18 located at the base of first reaction tube 4, but a person of ordinary skill in the art will appreciate that chiller 18 may be located at any position along first reaction tube 4 suitable to lower and/or maintain the fluid at the first temperature in first reaction tube 4.

Additionally, or alternatively, reactor 2 may comprise optional heater 20, that raises the temperature of the fluid to a second temperature suitable to facilitate decomposition of the endoperoxide and/or release of the stored oxygen, which than can leave reactor 2 through an oxygen outlet 22. Heater 20 may be located at any suitable position along second reaction tube 6, such as at the base of second reaction tube 6, as shown in FIG. 7. While FIG. 7 illustrates exemplary locations for optional chiller 18 and heater 20 that are within a fluid flow path in reactor 2, a person of ordinary skill in the art will appreciate that the chiller and/or heater alternatively may be located outside the respective reaction tubes thereby heating or cooling the fluid through the walls of the tubes. Suitable heating and/or cooling devices include, but are not limited to, a heat exchange device. Heating devices such as resistors, filaments, or photothermal materials may be used to heat in direct or indirect contact with the chamber. Cooling may achieved by passage of air or liquid by a fan or pump or by thermoelectric cooling.

Additionally, or alternatively, reactor 2 may comprise one or more agitators to facilitating mixing of the fluid in one or both of first and second reaction tubes 4 and 6, respectively, for example, by stirring. Such agitation may facilitate singlet oxygen formation and the reaction between single oxygen and the polycyclic aromatic compound.

As oxygen is released at least a portion of the polycyclic aromatic compound is regenerated in the fluid. The fluid comprising the regenerated polycyclic aromatic compound returns to first reaction tube 4 through second connecting tube 10, to complete the cycle. In some embodiments, the fluid within second reaction tube 6 is shielded from light, such as by having the walls of second reaction tube 6 being opaque so that the fluid within is maintained in the dark. This substantially prevents singlet oxygen formation from the released oxygen, and further endoperoxide formation.

C. Oxygen Concentrator

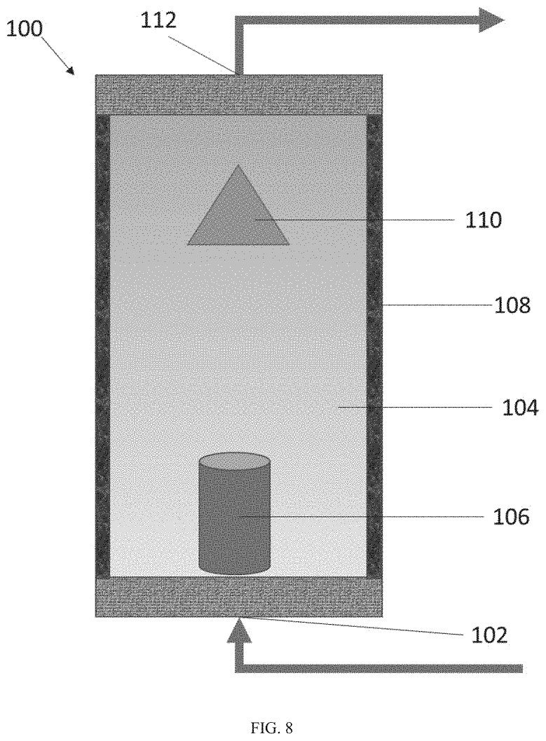

Certain embodiments of the disclosed technology concern an oxygen concentrator. FIG. 8 provides an exemplary embodiment of an oxygen concentrator. With respect to FIG. 8, the concentrator comprises chamber 100 that comprises a gas intake 102 through which a gas, such as air, enters chamber 100 and is exposed to media 104. Media 104 comprises polycyclic aromatic compound, such as a naphthalene and/or anthracene compound, and a photosensitizer. Media 104 may be a liquid media, such as a solution of the polycyclic aromatic compound and photosensitizer, or media 104 may be a solid media, and in some embodiments, is a porous solid media, such as a powder, crystalline structure, nanostructure, and/or granule.

Chamber 100 optionally may comprise a pump 106, to facilitate gas intake, and/or to compress the gas in the chamber to further facilitate oxygen uptake by media 104. FIG. 8 shows pump 106 located within chamber 100, but a person of ordinary skill in the art will understand that pump 106 could be located outside chamber 100 and be fluidly coupled to air intake 102. In some embodiments, pump 106 is located inside chamber 100 and draws the gas in to the chamber through gas intake 102. In such embodiments, gas intake 102 may be a gas permeable membrane, porous material, valve, or an opening to the gas, such as ambient air.

Chamber 100 further comprises one or more light sources 108. Light source(s) 108 may be located within one or more walls of chamber 100, as illustrated in FIG. 8. Additionally, or alternatively, light source(s) 108 may be located within chamber 100 but not in the walls, of the chamber. Or, light source(s) 108 may be windows in the walls of chamber 100, or windows that make up the walls of chamber 100, that facilitate light entering the chamber from an external light source, such as sunlight, and/or external artificial light (not shown). The solid support may be transparent to the wavelength of light exciting the triplet photosensitizer such as diatomaceous earth at visible-NIR wavelengths. And/or the pathlength of the solid support may be minimized to increase the transparency, such as with polymers. Additionally. or alternatively, the solid support may absorb the light and act as a singlet oxygen photosensitizer such as with nanoparticles.

Chamber 100 also may comprise a heating and/or cooling device. In some embodiments, the heating and/or cooling device is a single device, for example element 110 in FIG. 8. Alternatively, chamber 100 may comprise a separate heater and cooler to separate heat and cool the chamber. Suitable heating and/or cooling devices include, but are not limited to, a heat exchange device. Heating devices such as resistors, filaments, or photothermal materials may be used to heat in direct or indirect contact with the chamber. Cooling may achieved by passage of air or liquid by a fan or pump or by thermoelectric cooling.

During operation, a gas, such as air, enters chamber 100 through gas intake 102 and is exposed to the media. Light source(s) 108 provide light of a suitable wavelength to facilitate oxygen uptake by the media and endoperoxide formation. The remaining gas may be expelled from chamber 100 through outlet 112. In alternative embodiments, the remaining gas is released though gas intake 102, the gas intake being configured to allow flow in both directions.

Once a desired amount of oxygen has been stored by media 104, the concentrator may be configured to release oxygen. Typically, light source(s) 108 are turned off, removed or blocked, and/or heating/cooling device 110 is configured to heat media 104, thereby facilitating endoperoxide degradation and oxygen release. In some embodiments, gas intake 102 continues to facilitate gas entering chamber 100. The release oxygen mixes with the gas, for example air, forming an oxygen enriched gas mixture that leaves chamber 100 through outlet 112. In some embodiments, outlet 112 is connected to a breathing apparatus to provide oxygen-enriched air to a subject in need thereof.

In some embodiments, the oxygen concentrator comprises a single chamber 100, but other embodiments comprise two or more, such as two, three, four, five or more, chambers. In certain embodiments, a portable oxygen concentrator comprises two chambers; a first chamber being configured to store oxygen w % bile the second chamber is configured to release oxygen. Once the second chamber has released substantially all of the stored oxygen and/or is unable to provide oxygen enriched gas having a desired oxygen content, the second chamber is re-configured to store oxygen while the first chamber is re-configured to store oxygen. In some embodiments comprising two or more chambers, the chambers may also comprise a pump in each chamber. However, in alternative embodiments, a system comprising two or more chambers may comprise less than one pump per chamber. For example, a system comprising two chambers may comprise a single pump configured to pump to the first chamber or the second chamber separately, or configured to pump to both chambers simultaneously.

The disclosed system overcomes many of the challenges currently faced by the existing oxygen purification and/or storage solutions, as it is mainly be powered by sunlight or LEDs for oxygen consumption. Oxygen release can be initiated at near room temperature or increased with slightly higher temperatures above 25° C. Storage of the consumed oxygen is achieved by keeping the solution at lower temperatures around 16° C. and/or in a sealed container.

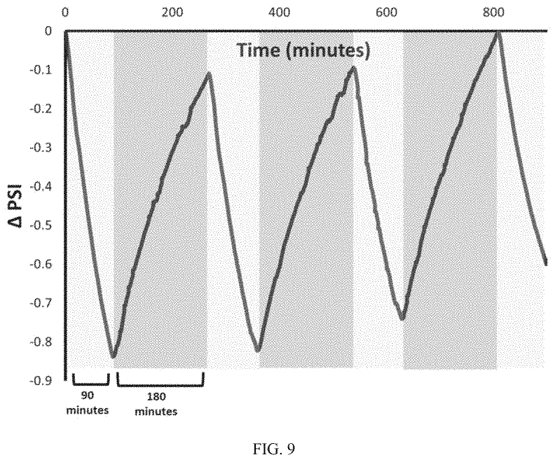

The utility of the system described herein is determined by its longevity-to repeat cycles of oxygen storage and release. After 4 cycles of irradiation under an oxygen atmosphere, an apparent change in the rate of oxidation was seen, indicated by a slowing of the rate of pressure decrease from each cycle of irradiation (FIG. 9). Despite this, the similar rates of oxygen release indicated that the 1,4-DMN was stable. Otherwise, if nonreversible oxidation was occurring, the release of oxygen also would be slowed. Without being bound to a particular theory, the results may suggest that rose bengal photobleaching is occurring, that would result in less ¹O₂ production and therefore alter the rate of oxidation. However, this could be addressed by using alternative triplet photosensitizers, such as methylene blue, Eosin B, Ru(bpy)₃, methyl green, rubrene, a fullerene (alkyl, aryl, alkoxy—substituted or unsubstituted C₂₋₉₄) a fluorene (e.g. comprising 9,9-substituted, or 2,7-substitued fluorene) a nanoparticle (e.g. CdTe, ZnSe, SiNP, CNP, AuNP, BiNP, or encapsulated small molecule triplet photosensitizers to form nanoparticles attached or encapsulated to/by silica, proteins, or polymers), or a combination thereof.

### V1. Exemplary Embodiments

The following numbered paragraphs illustrate exemplary embodiments of the disclosed technology.

Paragraph 1. A method, comprising:

exposing a first mixture comprising a polycyclic aromatic compound and a photosensitizer to a first portion of oxygen gas and light at a first temperature to form a second mixture comprising an endoperoxide, the first temperature being selected to substantially prevent the endoperoxide from decomposing;

raising a temperature of the second mixture to a second temperature higher than the first temperature and/or stopping irradiation to facilitate decomposition of the endoperoxide to form a regenerated polycyclic aromatic compound and release oxygen; and

mixing the released oxygen with an air stream to form an oxygen enriched air stream.

Paragraph 2. The method of paragraph 1, wherein exposing the first mixture to oxygen gas comprises exposing the first mixture to ambient air.

Paragraph 3. The method of paragraph 1 or paragraph 2, wherein the method further comprises providing the oxygen enriched air stream to a patient in need thereof paragraph 4. The method of any one of paragraphs 1-3, further comprising maintaining the second mixture at or below the first temperature for a selected time period before raising the temperature of the second mixture to the second temperature.

Paragraph 5. The method of any one of paragraphs 1-4, further comprising exposing the regenerated polycyclic aromatic compound and the photosensitizer to a second portion to oxygen gas to reform at least a portion of the endoperoxide.

Paragraph 6. A method, comprising:

a) exposing a first mixture comprising a first polycyclic aromatic compound and a first photosensitizer to a first portion of oxygen gas and light from a first light source at a first temperature to form a second mixture comprising a first endoperoxide, the first temperature being selected to substantially prevent decomposition of the first endoperoxide:

b) raising a temperature of the second mixture to a second temperature that is higher than the first temperature suitable to decompose the first endoperoxide thereby regenerating at least a portion of the first polycyclic aromatic compound and forming a second portion of oxygen gas that is mixed with a first air stream to form a first oxygen enriched air stream;

c) exposing a third mixture comprising a second polycyclic aromatic compound and a second photosensitizer to a third portion of oxygen gas and light from a second light source at a third temperature to form a fourth mixture comprising a second endoperoxide, the third temperature being selected to substantially prevent decomposition of the second endoperoxide;

d) when the first endoperoxide has substantially decomposed, raising a temperature of the third mixture to a fourth temperature that is higher than the third temperature and selected to decompose the second endoperoxide, thereby regenerating at least a portion of the second polycyclic aromatic compound and forming a fourth portion of oxygen gas that is mixed with a second air stream to form a second oxygen enriched air stream; and

e) exposing the first polycyclic aromatic compound and the first photosensitizer to a fifth portion of oxygen gas and light from the first light source to reform at least a portion of the first endoperoxide.

Paragraph 7. The method of paragraph 6, wherein step b is performed contemporaneously with step c.

Paragraph 8. A method, comprising:

exposing a first mixture comprising a polycyclic aromatic compound, a photosensitizer and oxygen gas to light in an enclosed space having a first volume to form an endoperoxide by a reaction between the polycyclic aromatic compound and the oxygen gas, thereby changing a volume of the enclosed space to a second volume that is less than the first volume; and

removing the light to regenerate the polycyclic aromatic compound, release at least a portion of the oxygen gas into the enclosed space thereby changing the volume of the enclosed space to a third volume that is substantially the same as the first volume.

Paragraph 9. The method of paragraph 8, wherein changing the volume of the enclosed space moves a piston.

Paragraph 10. A method, comprising:

exposing a first mixture comprising a polycyclic aromatic compound, a photosensitizer and oxygen gas to light in an enclosed space having a first pressure to form an endoperoxide by a reaction between the polycyclic aromatic compound and the oxygen gas, thereby changing a pressure in the enclosed space to a second pressure that is less than the first pressure; and

removing the light to regenerate the polycyclic aromatic compound, release at least a portion of the oxygen gas into the enclosed space thereby changing the pressure of the enclosed space to a third pressure that is substantially the same as the first pressure.

Paragraph 11. The method of paragraph 10 wherein changing the pressure of the enclosed space results in work.

Paragraph 12. The method of any one of paragraphs 8-11, further comprising exposing the regenerated polycyclic aromatic compound to light to reform at least a portion of the endoperoxide, and then removing the light source to regenerate the polycyclic aromatic compound.

Paragraph 13. The method of any one of paragraphs 8-12, wherein exposing the first mixture to the light is performed at a first temperature and removing the light further comprises raising the temperature to a second temperature higher than the first temperature.

Paragraph 14. The method of any one of paragraphs 1-13, wherein the polycyclic aromatic compound is a naphthalene compound or an anthracene compound.

Paragraph 15. The method of paragraph 14, wherein the polycyclic aromatic compound has a formula I

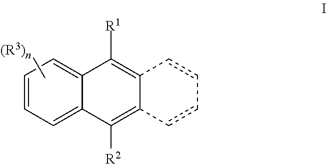

wherein:
each of R¹, R² and R³ independently is H, OH, aliphatic, aryl, alkoxy, —O-acyl, —O—Si(alkyl)₃, —O-amino acid, or —O-carbohydrate;

n is from 0 to 6; and

“---” represents a bond that may or may not be present.

Paragraph 16. The method of paragraph 15, wherein the compound has a formula selected from

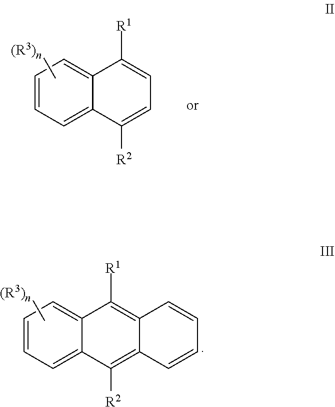

Paragraph 17. The method of paragraph 15 or paragraph 16, wherein n=0.

Paragraph 18. The method of any one of paragraphs 15-17 wherein at least one of R¹ and R² are not H.

Paragraph 19. The method of any one of paragraphs 15-18 wherein R¹ and R² are both alkyl.

Paragraph 20. The method of any one of paragraphs 15-19, wherein R¹ and R² are the same and not H.

Paragraph 21. The method of any one of paragraphs 15-19, wherein the polycyclic aromatic compound is 1,4-dimethylnaphthalene.

Paragraph 22. The method of any one of paragraphs 1-7 and 13-21, wherein the first temperature is from −78° C. to 25° C.

Paragraph 23. The method of paragraph 22, wherein the first temperature is from 0° C. to 20° C.

Paragraph 24. The method of any one of paragraphs 1-7 and 13-23, wherein the second temperature is from −40° C. to 100° C.

Paragraph 25. The method of paragraph 24, wherein the second temperature is from 15° C. to 25° C.

Paragraph 26. The method of any one of paragraphs 1-25, wherein the photosensitizer is rose bengal, methylene blue, Eosin B, Ru(bpy)₃, methyl green, rubrene, a fluorene, a fullerene, a nanoparticle or a combination thereof.

Paragraph 27. The method of paragraph 26, wherein:

the fullerene is a C₂₀₋₉₄ fullerene, optionally substituted with alkyl, aryl, alkoxy or a combination thereof:

the fluorene is 9,9-substituted fluorene or 2,7-substitued fluorene; and the nanoparticle is a CdTe, ZnSe, SiNP, CNP, AuNP, or BiNP nanoparticle, is a silica, protein or polymer nanoparticle that comprises a triplet photosensitizer, or a combination thereof.

Paragraph 28. The method of paragraph 26, wherein the photosensitizer is rose bengal.

Paragraph 29. The method of any one of paragraphs 1-28, wherein the light comprises light having a wavelength of from 380 nm to 1000 nm.

Paragraph 30. The method of paragraph 29, wherein the light is visible light.

Paragraph 31. The method of paragraph 30, wherein the visible light has a wavelength of from 380 nm to 740 nm.

Paragraph 32. The method of paragraph 31, wherein the visible light comprises light having a wavelength of from 530 nm to 575 nm.

Paragraph 33. The method of any one of paragraphs 1-21, wherein the light comprises natural light.

Paragraph 34. The method of paragraph 33, wherein the light is solar light.

Paragraph 35. The method of any one of paragraphs 1-33, wherein the light comprises light from an LED light source.

Paragraph 36. The method of any one of paragraphs 1-35, wherein the first mixture is a solution and further comprises a solvent.

Paragraph 37. The method of paragraph 36, wherein the solvent is acetonitrile, ethanol, methanol, dichloromethane, hexanes, water, ether, dimethylformamide, carbon tetrachloride, chloroform, propylene carbonate, ethylene glycol, propylene glycol, tetrahydrofuran or a combination thereof.

Paragraph 38. The method of paragraph 36, wherein the solvent is acetonitrile.

Paragraph 39. The method of any one of paragraphs 1-38, wherein the first mixture is a solid.

Paragraph 40. The method of paragraph 39, wherein the solid further comprises a zeolite, diatomaceous earth, hydrogel, polymer, metal-organic framework motif, or a combination thereof.

Paragraph 41. The method of paragraph 40, wherein the polymer comprises polyethylene, polypropylene, polyethylene glycol, polypropylene glycol, a peptide, a polyacrylic acid, or a combination thereof.

Paragraph 42. The method of paragraph 41, wherein the polymer has a molecular weight of from 5 kDa to 500 kDa.

Paragraph 43. A system, comprising:

a first chamber comprising a first mixture comprising a first polycyclic aromatic compound and a first photosensitizer, a first gas inlet, a first gas outlet, a first pump, and a first light source:

a second chamber comprising a second mixture of a first polycyclic aromatic compound and a second photosensitizer, a second gas inlet, a second gas outlet, a second pump, and a second light source; and

a controller configured to switch an air stream between the first and second gas inlets.

Paragraph 44. The system of paragraph 43, further comprising a temperature controller.

Paragraph 45. The system of paragraph 44, wherein the temperature controller is a heater, a cooler, or a combination thereof.

Paragraph 46. A system, comprising:

an enclosed space that can vary in volume and comprising a mixture comprising a polycyclic aromatic compound, a photosensitizer, and a gas comprising oxygen.

Paragraph 47. A system, comprising

an enclosed space that can vary in pressure and comprising a mixture comprising a polycyclic aromatic compound, a photosensitizer, and a gas comprising oxygen.

Paragraph 48. The system of paragraph 46 or paragraph 47, further comprising a light source.

In view of the many possible embodiments to which the principles of the disclosure may be applied, it should be recognized that the illustrated embodiments are only preferred examples of the technology and should not be taken as limiting the scope of the invention. Rather, the scope of the invention is defined by the following claims. We therefore claim as our invention all that comes within the scope and spirit of these claims.

We claim:

1. A method, comprising:
exposing a first mixture comprising a polycyclic aromatic compound and a photosensitizer to a first portion of oxygen gas and light to form a second mixture comprising an endoperoxide, wherein exposing the first mixture comprises one of options A, B, or C
A) exposing the first mixture at a first temperature to form the second mixture, the first temperature being selected to substantially prevent the endoperoxide from decomposing;
raising a temperature of the second mixture to a second temperature higher than the first temperature and/or stopping irradiation to facilitate decomposition of the endoperoxide to form a regenerated polycyclic aromatic compound and release oxygen; and
mixing the released oxygen with an air stream to form an oxygen enriched air stream;
B) exposing the first mixture in an enclosed space having a first volume to form the endoperoxide by a reaction between the polycyclic aromatic compound and the oxygen gas, thereby changing a volume of the enclosed space to a second volume that is less than the first volume; and
stopping irradiation by the light to regenerate the polycyclic aromatic compound, and release at least a portion of the oxygen gas into the enclosed space thereby changing the volume of the enclosed space to a third volume that is substantially the same as the first volume; or
C) exposing the first mixture in an enclosed space having a first pressure to form the endoperoxide by a reaction between the polycyclic aromatic compound and the oxygen gas, thereby changing a pressure in the enclosed space to a second pressure that is less than the first pressure; and
stopping irradiation by the light to regenerate the polycyclic aromatic compound, and release at least a portion of the oxygen gas into the enclosed space thereby changing the pressure of the enclosed space to a third pressure that is substantially the same as the first pressure.

2. The method of claim 1, wherein the method comprises option A and the method further comprises:
maintaining the second mixture at or below the first temperature for a selected time period before raising the temperature of the second mixture to the second temperature;
exposing the regenerated polycyclic aromatic compound and the photosensitizer to a second portion to oxygen gas to reform at least a portion of the endoperoxide; or
a combination thereof.

3. The method of claim 1, wherein the method comprises option A and the method comprises:
a) exposing a first mixture comprising a first polycyclic aromatic compound and a first photosensitizer to a first portion of oxygen gas and light from a first light source at a first temperature to form a second mixture comprising a first endoperoxide, the first temperature being selected to substantially prevent decomposition of the first endoperoxide;
b) raising a temperature of the second mixture to a second temperature that is higher than the first temperature suitable to decompose the first endoperoxide thereby regenerating at least a portion of the first polycyclic aromatic compound and forming a second portion of oxygen gas that is mixed with a first air stream to form a first oxygen enriched air stream;
c) exposing a third mixture comprising a second polycyclic aromatic compound and a second photosensitizer to a third portion of oxygen gas and light from a second light source at a third temperature to form a fourth mixture comprising a second endoperoxide, the third temperature being selected to substantially prevent decomposition of the second endoperoxide;
d) when the first endoperoxide has substantially decomposed, raising a temperature of the third mixture to a fourth temperature that is higher than the third temperature and selected to decompose the second endoperoxide, thereby regenerating at least a portion of the second polycyclic aromatic compound and forming a fourth portion of oxygen gas that is mixed with a second air stream to form a second oxygen enriched air stream; and
e) exposing the first polycyclic aromatic compound and the first photosensitizer to a fifth portion of oxygen gas and light from the first light source to reform at least a portion of the first endoperoxide.

4. The method of claim 1, wherein the method comprises option B and:
the method further comprises exposing the regenerated polycyclic aromatic compound to light to reform at least a portion of the endoperoxide, and then removing the light source to regenerate the polycyclic aromatic compound;
exposing the first mixture to the light is performed at a first temperature and removing the light further comprises raising the temperature to a second temperature higher than the first temperature; or
a combination thereof.

5. The method of claim 1, wherein the method comprises option C and:
the method further comprises exposing the regenerated polycyclic aromatic compound to light to reform at least a portion of the endoperoxide, and then removing the light source to regenerate the polycyclic aromatic compound;
exposing the first mixture to the light is performed at a first temperature and removing the light further comprises raising the temperature to a second temperature higher than the first temperature; or
a combination thereof.

6. The method of claim 1, wherein the polycyclic aromatic compound is a naphthalene compound or an anthracene compound.

7. The method of claim 6, wherein the polycyclic aromatic compound has a formula I 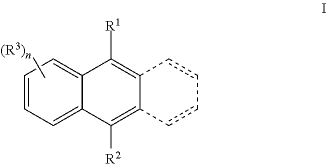
wherein:
each of R¹, R² and R³ independently is H, OH, aliphatic, aryl, alkoxy, —O-acyl, —O—Si(alkyl)₃, —O-amino acid, or —O-carbohydrate;
n is from 0 to 6; and
“---” represents a bond that may or may not be present.

8. The method of claim 7, wherein the compound has a formula selected from

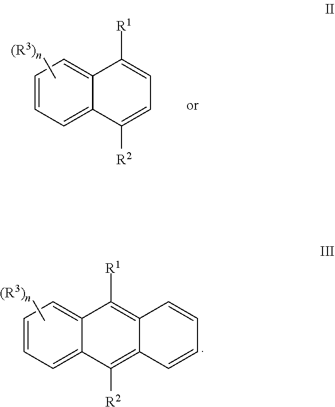

9. The method of claim 7, wherein n=0.

10. The method of claim 7, wherein:
at least one of R¹ and R² are not H;
R¹ and R² are both alkyl; or
R¹ and R² are the same and not H.

11. The method of claim 6, wherein:
the polycyclic aromatic compound is 1,4-dimethylnaphthalene;
the photosensitizer is rose bengal; or
a combination thereof.

12. The method of claim 6, wherein:
the first temperature is from −78° C. to 25° C.;
the second temperature is from −40° C. to 100° C.; or
a combination thereof.

13. The method of claim 12, wherein:
the first temperature is from 0° C. to 20° C.;
the second temperature is from 15° C. to 25° C.; or
a combination thereof.

14. The method of claim 1, wherein the photosensitizer is rose bengal, methylene blue, Eosin B, Ru(bpy)₃, methyl green, rubrene, a fluorene, a fullerene, a nanoparticle or a combination thereof.

15. The method of claim 14, wherein:
the fullerene is a C₂₀₋₉₄ fullerene, optionally substituted with alkyl, aryl, alkoxy or a combination thereof;
the fluorene is 9,9-substituted fluorene or 2,7-substitued fluorene; and
the nanoparticle is a CdTe, ZnSe, SiNP, CNP, AuNP, or BiNP nanoparticle, is a silica, protein or polymer nanoparticle that comprises a triplet photosensitizer, or a combination thereof.

16. The method of claim 1, wherein the light comprises light having a wavelength of from 380 nm to 1000 nm.

17. The method of claim 1, wherein:
the first mixture is a solution and further comprises a solvent; or
the first mixture is a solid.

18. The method of claim 17, wherein:
the solvent is acetonitrile, ethanol, methanol, dichloromethane, hexanes, water, ether, dimethylformamide, carbon tetrachloride, chloroform, propylene carbonate, ethylene glycol, propylene glycol, tetrahydrofuran or a combination thereof; and
the solid further comprises a zeolite, diatomaceous earth, hydrogel, polymer, metal-organic framework motif, or a combination thereof.

19. The method of claim 18, wherein the polymer comprises polyethylene,
polypropylene, polyethylene glycol, polypropylene glycol, a peptide, a polyacrylic acid, or a combination thereof.

20. A system, comprising:
a first chamber comprising a first mixture comprising a first polycyclic aromatic compound and a first photosensitizer, a first gas inlet, a first gas outlet, a first pump, and a first light source;
a second chamber comprising a second mixture of a first polycyclic aromatic compound and a second photosensitizer, a second gas inlet, a second gas outlet, a second pump, and a second light source; and
a controller configured to switch an air stream between the first and second gas inlets.
